(19) United States
(12) Patent Application Publication
(10) Pub. No.: US 2007/0042948 A1
(43) Pub. Date: Feb. 22, 2007

(54) Vav inhibition in graft rejection

(76) Inventors: Michael Forstner, Baerschwil (AT); Martin Klumpp, Lorrach (DE); Lukas Leder, Basel (CH); Barbara Nusslein-Hildesheim, Eimeldingen (DE); Friedrich Raulf, Freiburg (DE); Gisbert Weckbecker, Biel-Benken (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/552,836

(22) PCT Filed: Apr. 15, 2004
(86) PCT No.: PCT/EP04/03982

§ 371 Date: Oct. 13, 2005

(30) Foreign Application Priority Data
Apr. 16, 2003 (DE) 0308853.1
Jun. 27, 2003 (DE) 0315090.1

(51) Int. Cl. A61K 48/00; A61K 38/55; C12Q 1/68

(52) U.S. Cl. 514/12; 514/44; 435/6

(57) ABSTRACT

The present invention provides a method for preventing or treating acute or chronic graft rejection in a recipient of cell, tissue or organ allo- or xenotransplant, inflammatory or autoimmune diseases or malignant proliferative disease, comprising the use of an inhibitor of Vav. Further provided are Vav inhibitors, uses, pharmaceutical compositions and therapeutic combinations comprising Vav inhibitors, and screening methods therefore.

The present invention relates to transplantation and to promoting the viability of transplanted grafts, as well as to inflammatory and autoimmune diseases and malignant proliferative diseases. In one aspect, the invention relates to a method for inhibiting graft rejection (e.g. acute or chronic graft rejection).

More particularly, the invention relates to inhibiting graft rejection (e.g. acute or chronic graft rejection) by administering to a graft recipient a therapeutically effective amount of an inhibitor of a Vav protein.

Vav proteins include Vav1, Vav2 and Vav3. Vav1 is a 95 kDa signaling protein which was first identified in its oncogenic form during fibroblast transformation with human tumor DNA (Katzav et al. 1989, EMBO J. 8: 2283-2290). The sequence of Vav1 may be found under GenBank accession no. X16316, SwissProt accession no. P15498 and Ref. Seq no. NM_{—}005428. Vav1 is expressed exclusively in hematopoietic and trophoblast cells, and becomes rapidly phosphorylated on tyrosine in response to a variety of stimuli, including stimulation of TCR, B cell antigen receptor (BCR), and various cytokine receptors (Romero & Fischer, 1996, Cell. Signaling 8: 545-553; Collins et al. 1997, Immunol. Today 18: 221-225). Vav1 is also phosphorylated at serine 440 by protein kinase A, which suppresses Vav1 activation (WO 99/62315). Vav1 functions as a guanine-nucleotide exchange factor (GEF) for the RHO/RAC family of small GTPases, and regulates calcium mobilization, actin polymerization, receptor clustering and immune synapse formation in T-cells (Turner & Billadeau 2002, Nat. Rev. Immunol 2(7): 476-86). This reference and references cited therein also discuss Vav1^{-/-} and Vav2^{-/-} knockout mice. Vav2 and Vav3 are also GEFs but have broader patterns of expression.

Vav1 consists of a number of domains which are also found in Vav2 and Vav3. A DBL-homology (DH) domain interacts physically with Rac and Rho-GTPases and promotes the exchange of GTP for GDP. A SRC-homology 2 (SH2) domain is associated with recognition of phosphorylated tyrosine residues in receptor tyrosine kinases and adaptor proteins, leading to coupling of Vav1 to activated receptors. An acidic motif (Ac) is important for autoinhibition of Vav GEF activity. A calponin-homology (CH) domain is involved in regulation of Vav GEF activity and in calcium mobilization downstream of multi-subunit immune-recognition receptors. A pleckstrin-homology (PH) domain is involved in regulation of GEF activity by means of intramolecular interactions with the DH domain and by the binding of phophatidylinositols. A zinc-finger (ZF) domain is also involved in activation of GEF activity. A proline-rich (PR) region in Vav1 interacts with a SRC-homology 3 (SH3) domain in Vav1, such that the SH3 domain can bind to growth-factor-receptor-bound protein 2 (GRB2). Recognition of phosphorylated tyrosine residues by Vav1 SH2 domains can promote tyrosine phosphorylation of the Vav1 Ac domain, leading to activation of the DH domain GEF activity.

Preferably the inhibitor of a Vav protein is an inhibitor of Vav1. By “inhibitor of Vav1” is meant an agent or ligand (e.g. a molecule, a compound) which can inhibit a (i.e. one or more) function of the Vav1 protein. For example, the inhibitor may inhibit the binding of Vav-1 to a Rac and/or a Rho-GTPase, and/or inhibit the GEF activity of Vav-1. Alternatively, an inhibitor of Vav1 function may inhibit the binding of Vav1 to an activator of Vav-1 function and/or inhibit signal transduction mediated through Vav1. In one embodiment the inhibitor may inhibit the interaction of one or more SH2 domains in Vav1 with activated receptors, and/or inhibit the tyrosine phosphorylation of Vav1. In another embodiment, the inhibitor may interfere with the binding of the Vav1 SH3 domain to GRB2. Vav2 and Vav3 inhibitors may inhibit a function of Vav2 and Vav3 by analogous mechanisms.

Accordingly, Vav1 mediated processes and cellular responses (e.g., T and/or B cell activation, calcium mobilization, actin polymerization, receptor clustering, immune synapse formation in T cells) can be inhibited with an inhibitor of Vav1 function. As used herein, “Vav1” refers to naturally occurring Vav1, also known as Vav or p95^{vav}, (e.g. mammalian, preferably human (Homo sapiens) Vav1) and encompasses naturally occurring variants, such as allelic variants and splice variants, which retain Vav1 functional activity.

Preferably, the inhibitor of Vav1 function is a compound which is, for example, a small organic molecule, protein (e.g., antibody), peptide or peptidomimetic.

Example of proteins include e.g. antibodies, e.g., polyclonal, monoclonal, chimeric, humanized or human antibodies and antigen-binding fragments thereof (e.g., Fab, Fab′, F(ab′)₂, Fv): Examples of Vav1 binding antibodies are Vav (C-14), Vav (H-211), Vav (D-7), Vav (110-320); Vav (E-4), p-Vav (Tyr 174)-R, which are polyclonal or monoclonal antibodies raised against Vav1 or phophorylated Vav1, and which are available from Santa Cruz Biotechnology, Inc., Santa Cruz, Calif. Antigen-binding fragments can be produced by enzymatic cleavage or by recombinant techniques. For example, papain or pepsin cleavage can generate Fab or F(ab′)₂ fragments, respectively.

Other proteases with the requisite substrate specificity can also be used to generate Fab or F(ab′)₂ fragments. Antibodies can also be produced in a variety of truncated forms using antibody genes in which one or more Stop codons have been introduced upstream of the natural Stop site. For example, a chimeric gene encoding a F(ab′)₂ heavy chain portion can be designed to include DNA sequences encoding the CH, domain and hinge region of the heavy chain. Single-chain antibodies, and chimeric, human, humanized or primatized (CDR-grafted), or veneered antibodies, as well as chimeric, CDR-grafted or veneered single-chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., EP 0,125,023 B1; Boss et al., U.S. Pat. No. 4,816,397; Boss et al., EP 0,120,694 B1; Neuberger, M. S. et al., WO86/01533; Neuberger, M. S. et al., EP 0,194,276 B1; Winter, U.S. Pat. No. 5,225,539; Winter, EP 0,239,400 B1; Queen et al., EP 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242:423-426 (1988) regarding single-chain antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region. Using these or other suitable methods, variants can also be readily produced. In one embodiment, cloned variable regions can be mutated, and sequences encoding variants with the desired specificity can be selected.

Antibodies which are specific for mammalian (e.g., human) Vav1 can be raised against an appropriate immunogen, such as isolated and/or recombinant human Vav1 or portions thereof (including synthetic molecules, such as synthetic peptides). For example antibodies may be raised against a portion of Vav1 comprising an SH2 domain, or against a portion of Vav1 comprising the DH domain, or against phosphorylated or unphosphorylated Vav1. Antibodies can also be raised by immunizing a suitable host (e.g., mouse, rat) with cells that express Vav1, such as activated T cells (see, e.g., U.S. Pat. No. 5,440,020, the entire teachings of which are incorporated herein by reference). In addition, cells expressing recombinant Vav1 such as transfected cells, can be used as immunogens or in a screen for antibody which binds Vav1 (see, e.g., Chuntharapai et al., J. Immunol., 152; 1783-1789 (1994); U.S. Pat. No. 5,440,021).

Preparation of immunizing antigen, and polyclonal and monoclonal antibody production can be performed using any suitable technique. When a monoclonal antibody is desired, a hybridoma can generally be produced by fusing a suitable immortal cell line (e.g., a myeloma cell line such as SP2/0 or P3X63Ag8.653) with antibody-producing cells. The antibody-producing cells, preferably those obtained from the spleen or lymph nodes, can be obtained from animals immunized with the antigen of interest. The fused cells (hybridomas) can be isolated using selective culture conditions, and cloned by limiting dilution. Cells which produce antibodies with the desired specificity can be selected by a suitable assay (e.g. ELISA).

Other suitable methods of producing or isolating antibodies of the requisite specificity can be used, including, for example, methods which select recombinant antibody from a library (e.g., a phage display library). Transgenic animals capable of producing a repertoire of human antibodies (e.g., XenoMouse™ (Abgenix, Fremont, Calif.)) can be produced using suitable methods.

The term “peptide”, as used herein, refers to a compound consisting of from about two to about ninety amino acid residues wherein the amino group of one amino acid is linked to the carboxyl group of another amino acid by a peptide bond. Preferred peptide sequences are short (e.g. 3 to 20 amino acids in length) and lipophilic, such that they can cross cell membranes to a sufficient extent. A peptide can be, for example, derived or removed from a native protein by enzymatic or chemical cleavage, or can be prepared using conventional peptide synthesis techniques (e.g., solid phase synthesis) or molecular biology techniques (see Sambrook, J. et al., Molecular Cloning: A Laboratory Manual, Cold Spring Harbor Press, Cold Spring Harbor, N.Y. (1989)). A “peptide” can comprise any suitable L- and/or D-amino acid, for example, common o-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitrulline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art and are disclosed in, for example, Green and Wuts, “Protecting Groups in Organic Synthesis”, John Wiley and Sons, 1991. The functional groups of a peptide can also be derivatized (e.g., alkylated) using art-known methods.

The inhibitor may inhibit the binding of Vav1 to a Rac and/or a Rho-GTPase, and/or inhibit the GEF activity of Vav1. Such inhibitors may comprise antibodies which interfere with Vav1 binding to a GTPase, or peptide sequences found in the DH domain of Vav1, or sequences in a Rac or Rho-GTPase which are involved in binding to Vav1. The inhibitor may alternatively be an allosteric inhibitor which binds to a site on Vav1 remote from the DH domain, and which prevents exerts an effect on the DH domain such that GEF activity is inhibited.

Suitable inhibitory peptides may also include peptides which interfere with the phosphorylation of Vav1 by binding to the SH2 domain of Vav1, thereby inhibiting Vav1 coupling to activated receptors. For instance, an inhibitory peptide may be a tyrosine-phosphorylated peptide sequence which mimics the sequence recognized by the SH2 domain of Vav1, e.g. a peptide comprising the sequence pTyr-Xaa-Glu-Pro, where Xaa is Met, Leu or Glu, provided that the inhibitory peptide does not itself lead to Vav1 tyrosine phosphorylation and/or activation. Such inhibitory peptides may include sequences found in receptor tyrosine kinases and adaptor proteins to which Vav1 binds, for instance tyrosine phosphorylated sequences derived from the cytoplasmic domains of such receptors, e.g. SH2-binding sequences found in the T cell receptor, FcεR receptor, IgM receptor, p145^{c-kit}, II-2 receptor, IFNα receptor, SLP76 (SH2-domain-containing leukocyte protein of 76 kDa), B-cell linker (BLNK; SLP65/BASH) or CD19. In an alternative embodiment, the inhibitor may be a peptide comprising a sequence present in the SH2 domain of Vav1, e.g a sequence in Vav1 which is capable of interacting with the sequence pTyr-Xaa-Glu-Pro and/or with a tyrosine-phosphorylated sequence in one of the above receptor tyrosine kinases/adaptor proteins and thereby blocking the binding of Vav1 to the activated receptor/adaptor protein. For example the inhibitor peptide may comprise a sequence found in residues 671-765 of the human Vav1 protein.

Alternatively the inhibitor may be a peptide which interferes with the binding of the Vav1 SH3 domain to GRB2, for instance a peptide comprising an SH3-binding domain found in GRB2 or a GRB2-binding domain found in the SH3 domain of Vav1, e.g as disclosed in WO 95/26983.

In a further embodiment, the inhibitor may be a peptide which interferes with the phosphorylation of Vav1 by mimicking a phosphorylation site on Vav1, e.g. the Tyr174 phosphorylation site, or which associates with the phosphorylation site. Alternatively the inhibitor may be an enhancer of serine phosphorylation of Vav1, e.g. at Ser440, e.g. as described in WO 99/62315.

In a further embodiment, the inhibitor may be a peptide comprising an autoinhibitory sequence, e.g. a sequence found in the Ac domain of Vav1 which inhibits activation of Vav1 provided that it is not tyrosine-phosphorylated.

Suitable peptide sequences for use in these embodiments may be identified by probing the interaction of Vav1 with other molecular entities with anti-Vav1 antibodies, e.g. as specifically identified above or produced by methods as discussed above. For example, an anti-Vav1 monoclonal antibody which specifically inhibits binding of Vav1 to a Rho-GTPase may be used to identify short peptide sequences in Vav1 (e.g. in the DH domain) which may be used as inhibitors of Vav1 function.

The term “peptidomimetic”, as used herein, refers to molecules which are not polypeptides, but which mimic aspects of their structures. For example, polysaccharides can be prepared that have the same functional groups as peptides which can inhibit Vav1. Peptidomimetics can be designed, for example, by establishing the three dimensional structure of a peptide agent in the environment in which it is bound or will bind to Vav1. The peptidomimetic comprises at least two components, the binding moiety or moieties and the backbone or supporting structure.

The binding moieties are the chemical atoms or groups which will react or form a complex (e.g., through hydrophobic or ionic interactions) with Vav1, for example, with the amino acid(s) at or near the ligand binding site. For example, the binding moieties in a peptidomimetic can be the same as those in a peptide inhibitor of Vav1. The binding moieties can be an atom or chemical group which reacts with Vav1 in the same or similar manner as the binding moiety in a peptide inhibitor of Vav1. Examples of binding moieties suitable for use in designing a peptidomimetic for a basic amino acid in a peptide are nitrogen containing groups, such as amines, ammoniums, guanidines and amides or phosphoniums. Examples of binding moieties suitable for use in designing a peptidomimetic for an acidic amino acid can be, for example, carboxyl, lower alkyl carboxylic acid ester, sulfonic acid, a lower alkyl sulfonic acid ester or a phosphorous acid or ester thereof.

The supporting structure is the chemical entity that, when bound to the binding moiety or moieties, provides the three dimensional configuration of the peptidomimetic. The supporting structure can be organic or inorganic. Examples of organic supporting structures include polysaccharides, polymers or oligomers of organic synthetic polymers (such as polyvinyl alcohol or polylactide). It is preferred that the supporting structure possess substantially the same size and dimensions as the peptide backbone or supporting structure. This can be determined by calculating or measuring the size of the atoms and bonds of the peptide and peptidomimetic. In one embodiment, the nitrogen of the peptide bond can be substituted with oxygen or sulfur, thereby forming a polyester backbone. In another embodiment, the carbonyl can be substituted with a sulfonyl group or sulfinyl group, thereby forming a polyamide (e.g., a polysulfonamide). Reverse amides of the peptide can be made (e.g., substituting one or more —CONH— groups for a —NHCO— group). In yet another embodiment, the peptide backbone can be substituted with a polysilane backbone.

These compounds can be manufactured by known methods. For example, a polyester peptidomimetic can be prepared by substituting a hydroxyl group for the corresponding α-amino group on amino acids, thereby preparing a hydroxyacid and sequentially esterifying the hydroxyacids, optionally blocking the basic and acidic side chains to minimize side reactions. An appropriate chemical synthesis route can generally be readily identified upon determining the desired chemical structure of the peptidomimetic.

Peptidomimetics can be synthesized and assembled into libraries comprising a few to many discrete molecular species. Such libraries can be prepared using well-known methods of combinatorial chemistry, and can be screened as described herein to determine if the library comprises one or more peptidomimetics which inhibit Vav1 function. Such peptidomimetic antagonists can then be isolated by suitable methods.

Small organic molecules that can inhibit one or more functions of Vav1 may be identified by screening organic compounds from a library using a screening method as described below. A suitable small organic molecule may inhibit the function of Vav1 by any mechanism discussed above, e.g. by inhibiting the binding of Vav1 to a receptor tyrosine kinase, by mimicking autoinhibition by the Ac domain, by stabilising the autoinhibited form or preferably by inhibiting the binding of Vav1 to a Rho or Rac GTPase and/or by inhibiting the GEF activity of Vav1.

The capacity of an agent (e.g., proteins, peptides, natural products, small organic molecules, peptidomimetics) to inhibit Vav1 function can be determined using a suitable screen (e.g., high through-put assay). Preferably the assay comprises a step of determining the effect of an agent to be tested on the rate of guanine nucleotide exchange, e.g. the rate of exchange of GTP for GDP in (i.e. bound to) a suitable GTPase, e.g. RhoA or Rac GTPase, in the presence of Vav1. A decrease in the rate of guanine nucleotide exchange in the presence of the agent is indicative of a Vav1 inhibitor. Preferred Vav1 inhibitors may be identified as those agents which reduce the rate of guanine nucleotide exchange by at least 10%, more preferably at least 50%, most preferably at least 90%.

Vav1 may be activated by e.g. phosphorylation by an appropriate kinase, or by cleavage of the autoinhibitory Ac domain. Alternatively, the Vav1 protein used may show sufficient constitutive activity for the operation of the assay without a separate activation step. For example, the method may comprise using a constitutively active fragment of Vav1, e.g. comprising the DH domain, e.g. residues 181-375 of human Vav1, or a constitutively active mutant of Vav1, e.g. a Y174F mutant in which the tyrosine residue at position 174 is replaced by phenylalanine. Any fragment or mutant of Vav1 may be used, provided that it is active, i.e. it is capable of being activated to show GEF activity substantially similar to that shown by full-length Vav1.

For example, a Rac-1 GTPase may be loaded with fluorescently-labeled GDP (e.g. a BODIPY-labelled GTP/GDP analog available from Molecular Probes). The rate of loss of fluorescence is then measured in the presence of GTP and Vav1, or in the presence of GTP, Vav1 and an agent to be tested. If GTP only is added the fluorescence slowly decreases as GTP exchanges with fluorescent-GDP in the GTPase. Vav1 increases the rate of fluorescence loss by promoting exchange of GTP for GDP. An inhibitor of Vav1 reduces the rate of fluorescence loss relative to the rate in the presence of Vav1 and GTP. Preferred Vav1 inhibitors may be identified as those agents which reduce the rate of fluorescence loss by at least 10%, more preferably at least 50%, most preferably at least 90%.

Alternatively, the rate of increase of fluorescence when a fluorescent-labelled GDP is added to a GTPase may be measured in the presence of Vav1, and compared with the rate of increase in the presence of Vav1 and an agent to be tested. Vav1 increases the rate of fluorescence increase. An inhibitor of Vav1 reduces the rate of fluorescence increase. Preferred Vav1 inhibitors may be identified as those agents which reduce the rate of fluorescence increase by at least 10%, more preferably at least 50%, most preferably at least 90%.

The fluorescent-labelled GDP analog may be replaced in an alternative embodiment by fluorescent-labelled GTP. The fluorescent labeled GTP is cleaved by the GTPase to produce fluorescent-labelled GDP, the exchange of which may then be measured with unlabelled GTP. In a further alternative embodiment, the GTPase may be loaded with unlabelled GDP, fluorescent-labelled GTP added and a fluorescence increase measured in the presence of Vav1 In the presence of an inhibitor the rate of fluorescence increase is reduced.

In one embodiment the assay comprises the following steps:

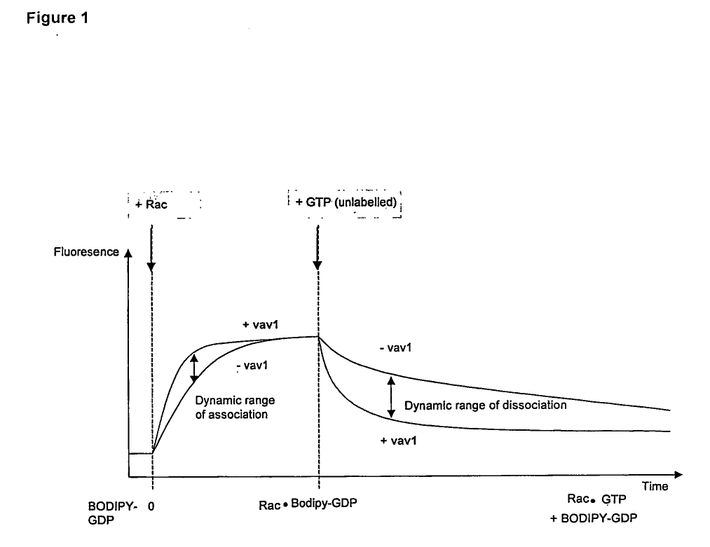

A fluorescently-labelled GDP (e.g. a BODIPY-labelled GTP/GDP analog, available from Molecular Probes, e.g. BODIPY-GDP) is incubated together with the GTPase Rac1 (produced as described below). The binding of the nucleotide to Rac1 leads to an increase in fluorescence, because the intra-molecular quenching of the fluorescence label by the guanine base is overcome when the latter becomes stuck to the binding pocket on the GTPase. This binding reaction can be accelerated by inclusion of an appropriate guanine nucleotide exchange factor (GEF) such as Vav1 in the reaction mixture (see FIG. 1).

Adding an excess of unlabelled GTP results in displacement of the fluorescent GDP from the GTPase with a concomitant decrease in fluorescence. This exchange reaction is accelerated by the nucleotide exchange factor Vav1 (see FIG. 1).

Vav1 activity can be determined from the time constant of the exchange reaction, or alternatively by comparing the fluorescence intensity at an appropriately chosen time-point in samples with and without Vav1. Vav1 inhibitors are detectable because they block the catalytic effect of the nucleotide exchange factor. Thus in the presence of an inhibitor the shape of the curve FIG. 1 shifts from that shown for “+Vav1” towards that shown for “−Vav1”.

Rac1 and Vav1 for use in the above method may be obtained by standard techniques such, as cloning, expression of the recombinant proteins and purification.

In one embodiment, Rac1 is prepared as follows. An insert coding for full length (aa 2-192) human Rac1 (RefSeq Nr. NM_{—}006908) is cloned into standard expression plasmids derived from the pET series (Novagen) with N-terminal tags like thioredoxin, GST, His₆-tag for affinity purification and cleavage site for PreScission Protease (APBiotech). The insert coding is located C-terminally to all the above mentioned tags. Expression is carried out in different E. coli strains like BL(21) DE3 Tuner (Novagen), whose DE3 element contains the DNA for T7 RNA polymerase enabling a high expression level of recombinant protein. Expression is carried out under standard conditions in LB medium, induction with IPTG and for various incubation times and temperatures. Bacteria are grown in 2 l Erlenmeyer shaker flasks. Purification is performed with affinity chromatography (Ni-NTA for His₆-tag, GSH Sepharose for GST tagged protein) as the first step. Further purification may be done with classical chromatographic techniques like size exclusion, ion exchange, hydrophobic interaction etc. The protein is either used with its tags still attached, or the tags are removed by proteolytic cleavage and chromatographic separation of the cleaved tag.

In one embodiment, Vav1 for use in the above method is obtained as follows. An insert coding for full length (aa 2-845) human Vav1 is cloned into standard expression plasmid derived from the FastBac series (Invitrogen) with N-terminal tag GST and His₆ for affinity purification and cleavage site for PreScission protease (APBiotech). Alternatively MBP and NusA N-terminal tags may also be used. The insert is located C-terminally to all the above mentioned tags. The bacmid for generation of baculovirus is generated by recombination in E. coli and purified with large plasmid purification kit (Qiagen). Since the protein is expressed in baculovirus infected insect cells, as the first steps sufficient amounts of baculovirus have to be generated by infection of Sf21 monolayer insect cells. Once, desired amounts of virus are obtained, the protein is produced in Hi Five suspension insect cells, whereby cells are grown in spinner flasks or Wave Bioreactors. Purification is performed with affinity chromatography (Ni-NTA for His₆-tag, GSH Sepharose for GST tagged protein) as the first step. Further purification may be done with classical chromatographic techniques like size exclusion, ion exchange, hydrophobic interaction etc. The protein is either used with its tags still attached, or the tags are removed by proteolytic cleavage and chromatographic separation of the cleaved tag.

A polypeptide comprising the Vav1 DH domain, e.g. residues 181-375 of human Vav1, may be produced in E. coli using similar methods.

As an alternative assay, Vav1 inhibitors may be identified by measuring the ability of an agent to be tested to inhibit apoptosis of Vav1-containing cells (e.g. thymocytes) induced by anti-CD3 and anti-CD28 antibodies. This assay is based on the observation that Vav1^{-/+} thymocytes undergo apoptosis following stimulation with anti-CD3 and anti-CD28 antibodies, whereas Vav1^{-/-} thymocytes fail to apoptose. The assay has the advantage that only non-toxic, cell permeable agents are identified. The ability of a compound to be tested to inhibit apoptosis may be compared in Vav1^{-/+} and Vav1^{-/-} cells.

Further assay methods for identifying Vav1 inhibitors include methods based on the downstream cellular effects of Vav1 activation, e.g. assays of calcium mobilization, CD69 expression or actin polymerization, using EYFP-actin to measure cytoskeletal re-organization. Such assay methods may be used in conjunction with a guanine exchange assay method as described above in order to identify whether the agent is acting via inhibition of Vav1 or on downstream events 2.

In a further embodiment, the assay method for Vav1 inhibitors comprises a label-free liquid chromatography-mass spectrometry-based high-throughput screening method, e.g. SpeedScreen. Such a method allows identification of agents which bind to Vav1 by means of affinity selection followed by size-exclusion chromatography and microbore-liquid-chromatography/electrospray-ionization mass spectrometry.

The Vav1 inhibitors thus identified are suitable for use in a method of the invention.

As used herein, the term “graft” refers to organs and/or tissues and/or cells which can be obtained from a first mammal (or donor) and transplanted into a second mammal (or recipient), preferably a human. The term “graft” encompasses, for example, skin, eye or portions of the eye (e.g., cornea, retina, lens), muscle, bone marrow or cellular components of the bone marrow (e.g., stem cells, progenitor cells), heart, lung, heartlung, liver, kidney, pancreas (e.g., islet cells, β-cells), parathyroid, bowel (e.g., colon, small intestine, duodenum), neuronal tissue, bone and vasculature (e.g., artery, vein). A graft can be obtained from suitable mammal (e.g., human or pig), or under certain circumstances a graft can be produced in vitro by culturing cells, for example embryonal, skin or blood cells and bone marrow cells. A graft is preferably obtained from human.

Organ transplants of kidney, heart, liver and lung are now regularly performed as treatment for endstage organ disease. Allograft as well as xenograft transplants have been performed. However, because of problems with long-term chronic rejection, organ transplantation is not yet a permanent solution to irreversible organ disease. There is also a need for improved agents for treatment of acute rejection.

Chronic rejection, which manifests itself as progressive and irreversible graft dysfunction, is the leading cause of organ transplant loss, in some cases already after the first postoperative year. The clinical problem of chronic rejection is clear from transplantation survival times; about half of kidney allografts are lost by around 10 years after transplantation, and a similar value is observed in patients with a heart allograft.

Chronic rejection is considered as a multifactorial process in which not only the immune reaction towards the graft but also the response of the blood vessel wall in the grafted organ to injury (“response-to-injury” reaction) plays a role. The variant of chronic rejection with the worst prognosis is an arteriosclerosis-like alteration, also called transplant vasculopathy, graft vessel disease, graft atherosclerosis, transplant coronary disease, etc. This vascular lesion is characterized by migration and proliferation of smooth muscle cells under influence of growth factors, that are amongst others synthesized by endothelium. A contribution of blood-borne stem cells as precursors of neo-intimal tissue has recently been demonstrated. It appears to progress also through repetitive endothelial injury induced amongst others by host antibody or antigen-antibody complexes, through intimal proliferation and thickening, smooth muscle cell hypertrophy repair, and finally to gradual luminal obliteration. Also so-called non-immunological factors like hypertension, hyperlipidemia, hypercholesterolemia etc. play a role.

Chronic rejection appears to be inexorable and uncontrollable because there is no known effective treatment or prevention modality. Thus, there continues to exist a need for a treatment effective in preventing, controlling or reversing manifestations of chronic graft vessel diseases.

Autoimmune and inflammatory disease includes, e.g. rheumatoid arthritis, systemic lupus erythematosus, Hashimoto's thyroidis, multiple sclerosis, myasthenia gravis, diabetes type I or II and the disorders associated therewith, vasculitis, pernicious anemia, Sjoegren syndrome, uveitis, psoriasis, Graves ophthalmopathy, alopecia greata and others, allergic diseases, e.g. allergic asthma, atopic dermatitis, allergic rhinitis/conjunctivitis, allergic contact dermatitis, inflammatory diseases optionally with underlying aberrant reactions, e.g. inflammatory bowel disease, Crohn's disease or ulcerative colitis, intrinsic asthma, inflammatory lung injury, inflammatory liver injury, inflammatory glomerular injury, atherosclerosis, osteoarthritis, irritant contact dermatitis and further eczematous dermatitis, seborrhoeic dermatitis, cutaneous manifestations of immunologically-mediated disorders, inflammatory eye disease, keratoconjunctivitis, myocarditis or hepatitis.

Malignant proliferative disease includes e.g. non-solid tumors, in particular leukemias and lymphomas, even more particular T-cell leukemias and T-cell lymphomas. T-cell leukemias and lymphomas include but are not limited to T-cell prolymphotic leukemia, T-cell granular lymphotic leukemia, aggressive NK cell leukemia, hairy-cell leukemia, nasal and nasal-type NK/T cell lymphoma, mycosis fungoides and Sezary syndrome, angioimmunoblastic T-cell lymphoma, peripheral T-cell lymphoma unspecified, adult T-cell leukemia/lymphoma (HTLV1+), anaplastic large cell lymphoma, primary cutaneous CD-30 positive T-cell lymphoproliferative disorders, cutaneous T-cell lymphoma, subcutaneous panniculitis like T-cell lymphoma, intestinal T-cell lymphoma (+enteropathy), and hepatosplenic gamma/delta T-cell lymphoma.

In accordance with the particular findings of the present invention, there is provided:
1.1 A method for preventing or treating acute graft rejection in a recipient of cell, tissue or organ allotransplant, comprising the step of administering to said recipient a therapeutically effective amount of an inhibitor of a mammalian (e.g. human) Vav protein;
1.2. A method of preventing or treating chronic rejection, e.g. to avoid, reduce or restrict chronic rejection, in a recipient of tissue or organ allotransplant, comprising the step of administering to said recipient a therapeutically effective amount of an inhibitor of a mammalian (e.g. human) Vav protein;
1.3. A method of preventing or treating graft vessel diseases, e.g. transplant vasculopathy, arteriosclerosis or atherosclerosis, in a recipient of tissue or organ allotransplant, comprising the step of administering to said recipient a therapeutically effective amount of an inhibitor of a mammalian (e.g. human) Vav protein;
1.4 A method of preventing or treating vein graft stenosis, restenosis and/or vascular occlusion following vascular injury, e.g. caused by catherization procedures or vascular scraping procedures such as percutaneous transluminal angioplasty, in a subject in need thereof, comprising administering to, said subject a therapeutically effective amount of an inhibitor of mammalian (e.g. human) Vav protein;
1.5 A method for preventing or treating an inflammatory or autoimmune disease in a subject in need thereof, comprising the step of administering to said subject a therapeutically effective amount of an inhibitor of a Vav protein;
1.6 A method for preventing or treating a malignant proliferative disease in a subject in need thereof, comprising the step of administering to said subject a therapeutically effective amount of an inhibitor of a Vav protein;
1.7 Use of an inhibitor of a Vav protein for preventing, treating or inhibiting (a) acute or chronic graft rejection in a recipient of cell, tissue or organ allotransplant, (b) vein graft stenosis, restenosis and/or vascular occlusion following vascular injury or (c) an inflammatory, autoimmune or malignant proliferative disease;
1.8 Use of an inhibitor of a Vav protein for the preparation of a medicament for preventing, treating or inhibiting (a) acute or chronic graft rejection in a recipient of cell, tissue or organ allotransplant, (b) vein graft stenosis, restenosis and/or vascular occlusion following vascular injury or (c) an inflammatory, autoimmune or malignant proliferative disease.
1.9. A pharmaceutical composition for use in any method as defined under 1.1 to 1.6 above comprising an inhibitor of a mammalian (e.g. human) Vav protein (e.g. an inhibitor of Vav1), together with one or more pharmaceutically acceptable diluents or carriers therefor.

The utility of Vav-1 as a target for inhibition in the prevention or treatment of transplant rejection is demonstrated in studies in transgenic Vav-1 deficient mice. The following method may be repeated, e.g. using Vav-2 deficient mice.

A.1 Heterotopic Vascular Heart Allotransplantation

Heterotopic heart transplantation is performed as described by Corry et al. 1973, Transplanation 16 (4), 343-350, using Balb/c (H-2d, Charles River, Wiga) mice as donors and Vav-1 ko mice and wildtype mice as recipients, which comprises an entire MHC and mH mismatch. Vav-1 ko knockout mice are generated as described in Fischer et al. 1998, Curr. Biol. 8, 554-562. Briefly, animals are anaesthetised using isoflurane. Following heparinisation of the donor mouse through the abdominal inferior vena cava with simultaneous exsanguination via the aorta, the chest is opened and the heart rapidly cooled. The aorta is ligated and divided distally to the first branch and the right brachiocephalic trunk is divided at the first bifurcation. The left pulmonary artery is ligated and divided and the right side divided. All other vessels are dissected free, ligated and divided and the donor heart is removed into iced saline. The recipient is prepared by dissection and cross-clamping of the infra-renal abdominal aorta and vena cava. The graft is implanted with end-to-side anastomoses, using 11/0 monofilament suture, between the donor right brachiocephalic trunk and the recipient aorta and the donor right pulmonary artery to the recipient vena cava. The clamps are removed, the graft tethered retroabdominally, the abdominal contents washed with warm saline and the animal is closed and allowed to recover under warming.

Grafts are monitored by daily palpation of the abdomen, and are considered rejected when there are no palpable ventricular contractions.

A.3 Treatment Groups

Animals are randomly assigned to one of the following treatment groups (n=5 per group):

(a) Balb/c-to-Vav-1 knockout;

(b) Balb/c-to-Vav-1 wild-type (C57BL/6).

TABLE 1
Cardiac allograft survival

| | | Median survival |
|---|---|---|
| Group | Graft survival time (days) | time (days) |
| Balb/c to Vav-1 +/+ | 7, 7, 8, 8, 8 | 8 |
| Balb/c to Vav-1 −/− | 50, 59, >100, >100, >100 | >100 |

Cardiac allograft survival is markedly increased in Vav-1 deficient mice compared to wild-type controls (shown in Table 1).

A.5.1 Histopathology and Immunohistochemistry

The degree of vascular remodelling for cardiac allografts with graft survival >100 days is assessed by scoring the remodelling of coronary vessels.

The tissue is fixed in 4% buffered formalin and embedded in paraffin; 3-4 μm thick sections of the graft are stained with hematoxylin and eosin, and Van Gieson stain to visualize the internal elastic lamina. Interstitial cellular rejection is scored as no rejection, mild, moderate or severe rejection. Vascular intimal thickening is scored as absent, mild, moderate or severe.

Immunohistochemical analysis is performed on paraffin sections. Streptavidin/peroxidase reactions are performed using primary antibodies to smooth muscle actin (SMA, clone 1A4, DakoCytomation, Denmark, dilution 1:25), rat anti-human CD3 (T cells, clone CD3-12, Serotec, UK, dilution 1:200) and rat anti-mouse F4/80 (macrophages, clone CI:A3-1, Serotec, UK, dilution 1:100) and rat anti-mouse CD45R (B cells, clone RA3-6B2, Serotec, dilution 1:10000). Semiquantitative analysis of smooth muscle cells and of the infiltrated CD3+, F4/80+ and CD45+ cells is performed using a Leica DMR microscope.

Individual NI values are averaged and reported as the mean±SEM. Hypothesis testing on the means of the treatment groups is performed using one way analysis of variance and the probability values are reported. Statistical significance is considered at p<0.05.

A.5.2 Histology of Cardiac Allografts

Vav-1+/+ recipients show severe acute cellular rejection after only 7-8 days. Analysis of grafts of Vav-1−/− shows only mild interstitial rejection with survival >100 days.

A.6 FACS Analysis and Haematology of Blood from Transplanted Mice

During autopsy, whole blood is sampled in heparin-coated tubes for FACS analysis. Furthermore, 250 μl whole blood is sampled in EDTA-coated tubes containing 250 μl PBS w/o Ca²⁺/Mg²⁺, after mixing 500 μl Nucleic Acid Purification Lysis Solution (Applied Biosystems #4305895) is added, vortexed for 0.5 min, and frozen at −20° C. A third blood sample is taken for H¹ haematology.

Splenocytes and peripheral blood are prepared from B6 WT mice and Vav1 knockout mice using standard procedures and subjected to ex vivo stimulation for 16 h using 200 000 cells/well and various stimuli (shown in table 2). Cells are immunophenotyped and the degree of stimulation determined in terms of CD69 expression using 3- or 4-color flow cytometry.

When WT and Vav1-deficient murine splenocytes or peripheral blood lymphocytes are subjected to various stimuli, Vav1 deficient T cells (CD4+, CD8+) and Vav1-deficient B cells (B220+) show a markedly lower expression of the activation marker CD69, as measured by FACS. Similar results are obtained for T and B cells from spleen and peripheral blood. Vav1 deficiency is demonstrated to block T cell activation and T-dependent B cell activation.

TABLE 2
CD69 expression T and B-cells

| | WT | | VAV−/− | |
|---|---|---|---|---|
| Spleen cells | Average | SEM | Average | SEM |
| | CD4+ | | | |
| MED | 0.95 | 0.22 | 1.46 | 0.07 |
| ConA 4.0 μg/ml | 44.53 | 13.44 | 11.24 | 1.84 |
| PMA 15 ng/ml + | 74.88 | 4.80 | 63.19 | 3.46 |
| A-CD28 1 μg/ml | | | | |
| A-CD3 0.25 μg/ml | 23.46 | 6.22 | 3.38 | 4.41 |
| A-CD3 0.25 μg/ml + | 34.08 | 3.05 | 9.26 | 5.24 |
| A-CD28 1 μg/ml | | | | |
| A-CD3 0.25 μg/ml + | 35.29 | 3.65 | 9.02 | 5.08 |
| A-CD28 2.5 μg/ml | | | | |
| | B220+ | | | |
| MED | 11.50 | 1.85 | 11.53 | 2.01 |
| ConA 40 μg/ml | 67.75 | 3.63 | 38.31 | 7.32 |
| PMA 15 ng/ml + | 83.24 | 2.88 | 81.70 | 6.82 |
| A-CD28 1 μg/ml | | | | |
| A-CD3 0.25 μg/ml | 57.91 | 15.74 | 14.47 | 2.31 |
| A-CD3 0.25 μg/ml + | 64.63 | 11.94 | 21.23 | 3.44 |
| A-CD28 1 μg/ml | | | | |
| A-CD3 0.25 μg/ml + | 65.45 | 11.95 | 26.68 | 10.71 |
| A-CD28 2.5 μg/ml | | | | |

Utility of the inhibitors of mammalian (e.g. human) Vav proteins in acute or chronic rejection, as well as utility in treating diseases and conditions as hereinabove specified, may be demonstrated in animal tests for example in accordance with the methods hereinafter described, as well as in clinic where e.g. the transplanted organ or tissue may be submitted to regular biopsy controls and in the case of heart transplant additionally to ultrasound scanning.

B.1. Acute Rejection

For example, Balb/c (H-2d) mice are used as donor animals, and e.g. CBA (H-2k) mice as recipients. The heart is removed from the donors according to a known procedure and stored in cold saline (4° C.). The recipient animals are anaesthetised with isofluorane. Infrarenal abdominal aorta and inferior vena cava are exposed. Blood vessels are dissected free from the fascia for a length of 3-5 mm, ligating and dividing any small branches. Vessels are occluded, first proximally and then distally. An arteriotomy and venotomy is performed, and lumens are flushed with heparinised physiological saline. End-to-side aortic anastomosis and then end-to-side anastomosis of the donor right pulmonary to recipient inferior vena cava are performed. The distal ligature is removed, then the proximal ligature. The suture lines are checked for leakage. Then the graft is tethered retroabdominally. The abdomen is flooded with warm saline (37° C.) and the wound is closed. Graft function is monitored daily by palpation of the abdomen. Rejection is concluded when the graft stops beating.

The animals are subject to one of the following treatments: an inhibitor of Vav1 alone, or a combined inhibitor of Vav1 and Vav2. Improvements of graft function are obtained in this assay in animals treated with an inhibitor of Vav1, or a combined Vav1/Vav2 inhibitor.

B.2 Chronic Rejection

Donor mice (e.g. C3H) are anaesthetised with isofluorane and left and right carotid arteries are isolated and dissected free. The animals are exsanguinated and heparinised saline 100 I.U./ml, is flushed through to remove away any residual blood. Each carotid artery is then removed as close to distal and proximal bifurcations as possible, rinsed again: with heparinised saline and stored in saline at 4° C. Recipient mice (e.g. B6) are anaesthetised as above and the left internal carotid artery lateral to the trachea, is isolated. A proximal microvascular clip and a distal clip are applied. An appropriate length midway between the clips is resected and the graft put in place and trimmed to length. End-to-end anastomoses are performed. The distal clip is removed first, then the proximal, haemostasis is ensured and the skin closed. The animals are placed under an infrared light until fully recovered.

The animals are subject to one of the following treatments: an inhibitor of Vav1 alone, or a combined inhibitor of Vav1 and Vav2.

At 5 to 8 weeks the animals are sacrificed, the carotid are perfused with 2 ml cold phosphate-buffered saline (PBS) and then with 2 ml cold perfusion fixative (2.5% gluteraldehyde, 2% formalin in PBS 0.01M). The carotid arteries are then excised and stained for histological evaluation. Morphometric analysis includes the measurement of the thickness of the media and intima. A qualitative analysis of the morphological changes include a scoring for adventitia infiltration of mononuclear cells and necrosis (vacuolar degeneration, hypertrophy of cells), the number of smooth muscle cells (SMC) nuclei in the media, SMC necrosis and the intimal infiltration of mononuclear cells. Improvements of graft function are obtained in this assay in animals treated with an inhibitor of Vav1, or a combined Vav1/Vav2 inhibitor.

B.3 Angioplasty

Studies on angioplasty are done in the rat model of balloon catheter injury. Balloon catheterization is performed on day 0, essentially as described by Powell et al. (1989). Under Isofluorane anaesthesia, a Fogarty 2F catheter is introduced into the left common carotid artery to obtain a uniform de-endothelialization. The catheter is then removed, a ligature placed around the external carotid to prevent bleeding and the animals allowed to recover. 2 groups of e.g. 12 RoRo rats (400 g, approximately 24 weeks old) are used for the study: one control group and one group receiving an inhibitor of Vav1 and the rats are fully randomized. The inhibitor of Vav1 is administered by infusion starting 2 days before balloon injury (day -3) until the end of the study, 14 days after balloon injury. The rats are then anaesthetized with Isofluorane and perfused with 0.1 M phosphate buffered saline solution (PBS, pH 7.4) and then for 15 min with 2.5% glutaraldehyde in phosphate buffer (pH 7.4). Carotid arteries are then excised, separated from surrounding tissue and immersed in 0.1 M cacodylate buffer (pH 7.4) containing 7% saccharose and incubated overnight at 40° C. The following day the carotids are then embedded in Technovit 7100 according to the manufacturers recommendation. The cross-sectional area of the media, neointima and the lumen are evaluated morphometrically by means of an image analysis system (MCID, Toronto, Canada).

Inhibitors of a Vav protein (e.g. Vav1 inhibitors) may be administered as the sole active ingredient or together with other drugs in immunomodulating regimens or other anti-inflammatory agents e.g. for the treatment or prevention of allograft acute or chronic rejection, inflammatory or autoimmune disorders or malignant proliferative diseases. For example, they may be used in combination with calcineurin inhibitors, e.g. cyclosporine A, cyclosporine G, FK-506, ABT-281, ASM 981; an mTOR inhibitor, e.g. rapamycin, 40-O-(2-hydroxy)ethyl-rapamycin, CC1779, ABT578, AP23573, AP23464, AP23675, AP23841 or TAFA-93; corticosteroids; cyclophosphamide; azathioprine; methotrexate; an EDG receptor agonist, e.g. FTY 720 or an analogue thereof; leflunomide or analogs thereof; mizoribine; mycophenolic acid; mycophenolate mofetil; 15-deoxyspergualine or analogs thereof; immunosuppressive monoclonal antibodies, e.g., monoclonal antibodies to leukocyte receptors, e.g., MHC, CD2, CD3, CD4, CD11a/CD18, CD7, CD25, CD27, B7, CD40, CD45, CD58, CD137, ICOS, CD150 (SLAM), OX40, 4-1BB or their ligands, e.g. CD154; or other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4lg (for ex. designated ATCC 68629) or a mutant thereof, e.g. LEA29Y, or other adhesion molecule inhibitors, e.g. mAbs or low molecular weight inhibitors including LFA-1 antagonists, Selectin antagonists and VLA-4 antagonists; chemotherapeutic agents e.g. antineoplastic agents or agents used as adjuvants in cancer therapy, e.g. aromatase inhibitors, antiestrogens, topoisomerase I inhibitors, topoisomerase II inhibitors, microtubule active agents, alkylating agents, antineoplastic antimetabolites, platin compounds, compounds decreasing the protein kinase activity, in particular non-receptor tyrosine kinase and further anti-angiogenic compounds, gonadorelin agonists, anti-androgens, bisphosphonates, trastuzumab and miscellaneous anti-cancer agents, e.g. 6-thioguanidine, hydroxyurea, procarbazine or bleomycin; or an anti-T cell immunotoxin fusion protein comprising a diptheria or Pseudomonas toxin moiety and a targeting moiety suitable for targeting the fusion protein to T cells (e.g. an anti-CD3 antibody), e.g. DT389-sFv(UCHT1), scFv(UCHT1)-PE38 and (Ala)dmDT390-bisFv(UCHT1*) which can be prepared and administered as described in WO 01/87982, WO 00/41474 or U.S. patent application Ser. No. 09/573,797, the contents thereof being incorporated herein by reference, or a chimeric diphtheria immunotoxin as disclosed in EP 517829 and marketed under the tradename ONTAK.

In a further aspect, the inhibitor is an inhibitor of Vav2. By “inhibitor of Vav2” is meant an agent or ligand (e.g. a molecule, a compound) which can inhibit a (i.e. one or more) functions of Vav2. Thus the inhibitor may inhibit the GEF activity of Vav2, or by analogy with Vav1, any of the other functions of Vav2. Preferably, the inhibitor of Vav2 function is a compound which is, for example, a small organic molecule, protein (e.g., antibody), peptide or peptidomimetic. Examples of Vav2 binding antibodies are Vav2 (D-7) and Vav2 (P-18) available from Santa Cruz Biotechnology, Inc., Santa Cruz, Calif. Other anti-Vav2 antibodies may be produced by analogy to the methods described above for anti-Vav1 antibodies. Peptide, peptidomimietic and small organic molecule inhibitors of Vav2 may also be produced by analogous processes to those described above for Vav1 inhibitors. Vav2 inhibitors may be used in methods as described above in relation to Vav1.

Vav2 inhibitors may be identified by a guanine-nucleotide exchange assay as described above, except that Vav2 is used in place of Vav1. In a further aspect, the invention provides a therapeutic combination comprising a Vav1 inhibitor and a Vav2 inhibitor. Vav1 inhibitors may be further screened for Vav2 inhibitory activity in such an assay, in order to identify agents which are capable of inhibiting both Vav1 and Vav2. Thus in one aspect, the inhibitor is a combined Vav1 and Vav2 inhibitor, by which it is meant a single agent which is capable of inhibiting both Vav1 and Vav2. Use of a combined Vav1/Vav2 inhibitor, or combined administration of a Vav1 inhibitor and a Vav2 inhibitor, is preferred when treating conditions where inhibition of both T and B cell function, activation and/or proliferation is desired.

The amount of an inhibitor of a Vav protein or additional drug administered to the individual will depend on the characteristics of the individual, such as general health, age, sex, body weight, the compound employed, the mode of administration, the severity of the condition to be treated. The skilled person will be able to determine appropriate dosages depending on these and other factors. Typically, an effective amount can range from about 0.1 mg per day to about 100 mg per day for an adult. Antibodies and antigen-binding fragments thereof, particularly human, humanized and chimeric antibodies and antigen-binding fragments can often be administered less frequently than other types of therapeutics. For example, an effective amount of such an antibody can range from about 0.01 to 5 or 10 mg/kg administered daily, weekly, biweekly, monthly or less frequently.

The inhibitor of a Vav protein (e.g. Vav1 inhibitor) may be administered by any conventional route, in particular enterally, e.g. orally, for example in the form of solutions for drinking, tablets or capsules or parenterally, for example in the form of injectable solutions or suspensions, or topically. The inhibitor of Vav may be administered as a neutral compound or as a pharmaceutically acceptable salt, e.g. salts obtained with a suitable acid or base.

In accordance with the foregoing the present invention provides in a yet further aspect:
5. A method as defined above comprising co-administration, e.g. concomitantly or in sequence, of a therapeutically effective amount of an inhibitor of a Vav protein (e.g. a Vav1 inhibitor) and at least one second drug substance, said second drug substance being an immunosuppressant, immunomodulatory or anti-inflammatory drug, or a chemotherapeutic agent, e.g. as indicated above.
6. A therapeutic combination, e.g. a kit, comprising a) an inhibitor of a Vav protein (e.g. a Vav1 inhibitor) and b) at least one second agent selected from an immunosuppressant, immunomodulatory or anti-inflammatory drug, or a chemotherapeutic agent. Component a) and component b) may be used concomitantly or in sequence. The kit may comprise instructions for its administration.
7. A method as defined above comprising co-administration, e.g. concomitantly or in sequence, of a therapeutically effective amount of an inhibitor of Vav1 and an inhibitor of Vav2.
8. A therapeutic combination, e.g. a kit, comprising a) an inhibitor of Vav1 and b) an inhibitor of Vav2. Component a) and component b) may be used concomitantly or in sequence. The kit may comprise instructions for its administration.
9. A screening method for identifying an agent as an inhibitor of Vav1, comprising a step of determining the rate of guanine nucleotide exchange in a Rho or Rac GTPase in the presence of (a) the agent and (b) Vav1, or an active fragment or mutant thereof, wherein a decrease in the rate of guanine nucleotide exchange in the presence of the agent is indicative that the agent is an inhibitor of Vav1.
10. A screening method for identifying an agent as an inhibitor of Vav2, comprising a step of determining the rate of guanine nucleotide exchange in a Rho or Rac GTPase in the presence of (a) the agent and (b) Vav2, or an active fragment or mutant thereof, wherein a decrease in the rate of guanine nucleotide exchange in the presence of the agent is indicative that the agent is an inhibitor of Vav2.
11. A Vav1 or Vav2 inhibitor obtainable by a method as defined above.

FIG. 1 shows fluorescence changes over time in a guanine nucleotide exchange assay for Vav1 inhibitors, in the presence or absence of Vav1, using a Rac GTPase and BODIPY-GDP.

The invention will now be described more precisely with reference to the following non-limiting examples.

### EXAMPLE 1

Bodipy-GDP/Rac-1 Association Assay

To each well of a 1536 well microtitre plate is added:

| | Final concentration | |
|---|---|---|
| Amount added | (after addition of Rac-1) | |
| 2 pmol recombinant human Vav-1 protein | 1 | μM |
| 0.2 pmol Bodipy-GDP | 100 | μM |
| 40 pmol test compound | 20 | μM |

in 0.1 M phosphate buffer, pH 7.0. 0.4 pmol (final concentration 200 nM) recombinant human Rac-1 is added to each well simultaneously to start the reaction, to give a final volume of 2 μl in each well. A different test compound is added to each well. Control wells contain each of the above components but either (a) no test compound or (b) neither a test compound nor Vav-1.

The plate is incubated for 30 minutes at 25° C. Fluorescence is then measured simultaneously for each well in a fluorescence plate reader, using an excitation wavelength of 488 nm and measuring at 530 nm.

Measured fluorescence is highest in control wells (a) containing Vav-1 and no inhibitor. Lower (baseline) fluorescence is recorded in control wells (b) containing no Vav-1; Wells containing active inhibitors of Vav-1 as test compounds may be identified as they show lower fluorescence than control wells (a).

### EXAMPLE 2

Bodipy-GDP/Rac-1 Dissociation Assay

For loading of recombinant human Rac1 with fluorescently labeled guanine nucleotide, 32 μM of recombinant human Rac1 in 50 mM Tris-HCl pH 7.5, 1000 μl of 50 mM Tris-HCl pH 7.5, 60 mM NaCl, 6 mM MgCl₂, 8 mM DTT and 500 μl of 480 nM BODIPY-FL-GDP are combined and incubated for 3 hours at room temperature. Of this mixture, 600 nl are then added to each well of a 2080 well microtitre plate, in which 600 nl of 3 μM vav1, 400 μM GTP in 50 mM Tris-HCl pH 7.5 have already been pre-incubated with the compounds to be tested. Control wells either (a) contain no test compound or (b) neither contain a test compound nor Vav-1.

The plate is then incubated at 25° C. Fluorescence is determined using a confocal or non-confocal fluorescence reader in each well 30 minutes after the start of the reaction, by obtaining a fluorescence reading for each well in the same sequence as the reaction was started. Wavelengths of excitation/detection are as for example 1.

Measured fluorescence is lowest in control wells (a) containing Vav-1 and no inhibitor. Higher fluorescence is recorded in control wells (b) containing no Vav-1. Wells containing active inhibitors of Vav-1 as test compounds can be identified based on an increased fluorescence compared to control wells (a).

In further examples, the methods of examples 1 and 2 may be repeated at different concentrations of test compound (e.g. 10 μM, 50 μM), different temperatures (e.g. 37° C.) and different incubation times (e.g. 15 minutes, 120 minutes) in order to maximize the dynamic range of association or dissociation (the difference between fluorescence in control wells (a) and (b) at the time of measurement). Other plate formats may be used and both the association and dissociation assays may be performed using either simultaneous or sequential fluorescence detection. Inhibitors of Vav-2 may be identified using an analogous assay employing recombinant human Vav-2 instead of Vav-1.

In further embodiments, the above examples may be repeated using (a) a truncated, mutant of Vav-1 comprising the DH domain, or (b) a Y174F mutant in place of full-length Vav-1.

1. A method for preventing or treating (i) acute or chronic graft rejection in a recipient of cell, tissue or organ allo- or xenotransplant, (ii) an inflammatory or autoimmune disease in a subject in need thereof, (iii) vein graft stenosis, restenosis and/or vascular occlusion following vascular injury or (iv) a malignant proliferation disease in a subject in need thereof, comprising a step of administering to the recipient a therapeutically effective amount of an inhibitor of a Vav protein.

2. (canceled)

3. A method or use according to claim 1 or claim 2, wherein the Vav protein is Vav1.

4. A therapeutic combination comprising an inhibitor of a Vav protein and at least one second agent selected from an immunosuppressant, immunomodulatory or anti-inflammatory drug, or a chemotherapeutic agent.

5. A screening method for identifying an agent as an inhibitor of Vav1 or Vav2, comprising a step of determining the rate of guanine nucleotide exchange in a Rho or Rac GTPase in the presence of (a) the agent and (b) Vav1 or Vav2, or an active fragment or mutant thereof, wherein a decrease in the rate of guanine nucleotide exchange in the presence of the agent is indicative that the agent is an inhibitor of Vav1 or Vav2.

6. A screening method according to claim 5, comprising a step of measuring the rate of exchange of a fluorescent-labeled guanine nucleotide for an unlabelled guanine nucleotide in the GTPase in the presence of (a) the agent and (b) Vav1 or Vav2, or an active fragment or mutant thereof.

7. A screening method according to claim 6, wherein the method comprises
(i) incubating the GTPase with a fluorescent-labelled guanine nucleotide and
(ii) measuring fluorescence changes in the presence of
(a) the agent
(b) Vav1 or Vav2, or an active fragment or mutant thereof, and
(c) an unlabelled guanine nucleotide.

8. A screening method according to claim 6, which is suitable for high-throughput screening.

9. A Vav1 or Vav2 inhibitor obtainable by a screening method according to claim 6.

10. A pharmaceutical composition for use in a method as defined in claim 1 comprising an inhibitor of a Vav protein and one or more pharmaceutically acceptable diluents or carriers therefore.
